(19) United States
(12) Patent Application Publication
(10) Pub. No.: US 2026/0034375 A1
(43) Pub. Date: Feb. 5, 2026

(54) DEFIBRILLATOR

(71) Applicant: FUKUDA DENSHI CO., LTD., Tokyo (JP)

(72) Inventors: Taichi Iwasaki, Tokyo (JP); Ryosuke Sakai, Tokyo (JP); Katsuya Ide, Tokyo (JP); Koki Oya, Tokyo (JP); Takashi Yamaki, Tokyo (JP); Kohei Mitsumaru, Tokyo (JP); Hiroshi Hino, Tokyo (JP); Yuzuru Sakai, Tokyo (JP)

(21) Appl. No.: 19/281,784

(22) Filed: Jul. 28, 2025

(30) Foreign Application Priority Data
Jul. 31, 2024 (JP) 2024-124989

(51) Int. Cl. A61N 1/39; H03K 3/021

(52) U.S. Cl. CPC A61N 1/3981; A61N 1/3904; H03K 3/021

(57) ABSTRACT

A defibrillator of the present disclosure includes a H-bridge type biphasic pulse generation circuit connected to the rear stage side of a high-voltage capacitor. The biphasic pulse generation circuit includes a first switch, a second switch connected in parallel to the first switch, a third switch connected in series and to the rear stage side of the first switch, and a fourth switch connected in series and to the rear stage side of the second switch. The biphasic pulse generation circuit outputs a biphasic pulse from a first output line connected to a connection intermediate point between the first and third switches and from a second output line connected to a connection intermediate point between the second and fourth switches. In at least one of the first switch to the fourth switch, a plurality of thyristors are connected in series, and a resistor is connected in parallel to each thyristor.

### CROSS REFERENCE TO RELATED APPLICATIONS

This application is entitled to and claims the benefit of Japanese Patent Application No. 2024-124989, filed on Jul. 31, 2024, the disclosure of which including the specification, drawings and abstract is incorporated herein by reference in its entirety.

### TECHNICAL FIELD

The technology of the present disclosure relates to a defibrillator.

### BACKGROUND ART

A defibrillator is, for example, a medical device for removing fibrillation of a heart by applying an electric shock to a patient who has developed ventricular fibrillation and cannot pump blood throughout the body, thereby restoring movement of the heart to normal.

The defibrillator generates a high voltage internally, charges a capacitor with the

voltage, and then discharges the voltage to an electrode pad or paddle attached to a patient, thereby delivering an electric shock to the patient.

For example, the configuration of a discharge section in a defibrillator is described in PTL 1, PTL 2, or the like. Since the defibrillator discharges high voltage, a component capable of withstanding the high voltage is used in a switch that controls the discharge.

### CITATION LIST

### Patent Literature

PTL 1
Japanese Unexamined Patent Application Publication (Translation of PCT Application) No. 2020-503916
PTL 2
Japanese Unexamined Patent Application Publication (Translation of PCT Application) No. 2008-514330

The defibrillation voltage reaches a maximum of about 2000 [V]. Therefore, the discharge section that discharges the defibrillation voltage is configured of a component capable of withstanding a high voltage. In addition, when a biphasic pulse is output from the discharge section, the discharge section is configured to have a plurality of switching sections (herein a switching section is simply referred to as “switch”) capable of withstanding the high voltage, which complicates the configuration and increases the product cost.

In particular, since it is desired to manufacture as many products as possible defibrillators such as automated external defibrillators (AEDs) and widely spread defibrillators in society, it is required to reduce the product costs.

The present disclosure has been made in consideration of the above points, and an object of the present disclosure is to provide a defibrillator that can use a lower-voltage withstanding component than that in the related art while favorably maintaining an original operation as a defibrillator.

### SUMMARY OF INVENTION

One aspect of the defibrillator of the present disclosure is a defibrillator including a biphasic pulse generation circuit connected to a rear stage side of a high-voltage capacitor, the biphasic pulse generation circuit being of an H-bridge type, in which:
the biphasic pulse generation circuit includes
   a first switch,
   a second switch that is connected in parallel to the first switch,
   a third switch that is connected to a rear stage side of the first switch, the third switch being connected in series to the first switch, and
   a fourth switch that is connected to a rear stage side of the second switch, the fourth switch being connected in series to the second switch;
the biphasic pulse generation circuit outputs a biphasic pulse from a first output line connected to a connection intermediate point between the first switch and the third switch and from a second output line connected to a connection intermediate point between the second switch and the fourth switch; and
in at least one of the first switch to the fourth switch, a plurality of thyristors are connected in series and a resistor is connected in parallel to each of the plurality of thyristors.

One aspect of the defibrillator of the present disclosure is a defibrillator including a biphasic pulse generation circuit connected to a rear stage side of a high-voltage capacitor, the biphasic pulse generation circuit being of an H-bridge type, in which:
the biphasic pulse generation circuit includes
   a first switch,
   a second switch that is connected in parallel to the first switch,
   a third switch that is connected to a rear stage side of the first switch, the third switch being connected in series to the first switch, and
   a fourth switch that is connected to a rear stage side of the second switch, the fourth switch being connected in series to the second switch;
the biphasic pulse generation circuit outputs a biphasic pulse from a first output line connected to a connection intermediate point between the first switch and the third switch and from a second output line connected to a connection intermediate point between the second switch and the fourth switch;
at least one of the first switch to the fourth switch has a configuration in which a plurality of insulated gate bipolar transistors (IGBTs) are connected in series;
a plurality of branch signal lines branched from a common gate signal line from a controller are respectively connected to gates of the plurality of IGBTs; and
a signal delay amount of the branch signal line connected to an IGBT located on a front stage side among the plurality of IGBTs is larger than a signal delay amount of the branch signal line connected to an IGBT located on a rear stage side among the plurality of IGBTs.

### BRIEF DESCRIPTION OF DRAWINGS

FIG. 1 is a circuit diagram illustrating the schematic configuration of a defibrillator according to an embodiment;

FIG. 2 is a circuit diagram illustrating a configuration example of a defibrillator of a comparative example;

FIG. 3 is a diagram for describing ON/OFF operations of switches of a biphasic pulse generation circuit of the defibrillator;

FIG. 4 illustrates a configuration in which two thyristors are simply connected in series as a reference example;

FIGS. 5A and 5B illustrate the potential and voltage values when the thyristors are turned on and off in which the configuration of FIG. 4 is adopted;

FIG. 6 illustrates a configuration example 1 of a portion related to the thyristors of the present embodiment;

FIGS. 7A and 7B illustrate the potential and voltage values when the thyristors are turned on and off in which the configuration of FIG. 6 is adopted;

FIG. 8 illustrates a configuration example 2 of a portion related to the thyristors of the embodiment;

FIG. 9 is a circuit diagram when thyristor SCR1 is regarded as variable resistor r1 in the configuration example 2;

FIG. 10 is a diagram in which the configuration of FIG. 9 is rewritten;

FIG. 11 illustrates a state of voltage increase immediately after a deviation in ON timing in the configuration of the embodiment;

FIG. 12 illustrates an LCR series circuit;

FIG. 13 illustrates a time response of voltage sharing of the elements based on a solution of a differential equation;

FIG. 14 illustrates a time response of voltage sharing of the elements based on a solution of a differential equation;

FIG. 15 illustrates a configuration example of a portion related to IGBTs of the embodiment;

FIG. 16 illustrates a configuration example of a portion related to the IGBTs of the embodiment;

FIG. 17 is a circuit diagram illustrating the configuration of another embodiment; and

FIG. 18 is a circuit diagram illustrating the configuration of another embodiment.

### DESCRIPTION OF EMBODIMENTS

Hereinafter, an embodiment of the present disclosure will be described in detail with reference to the drawings.

### <1>Schematic Configuration

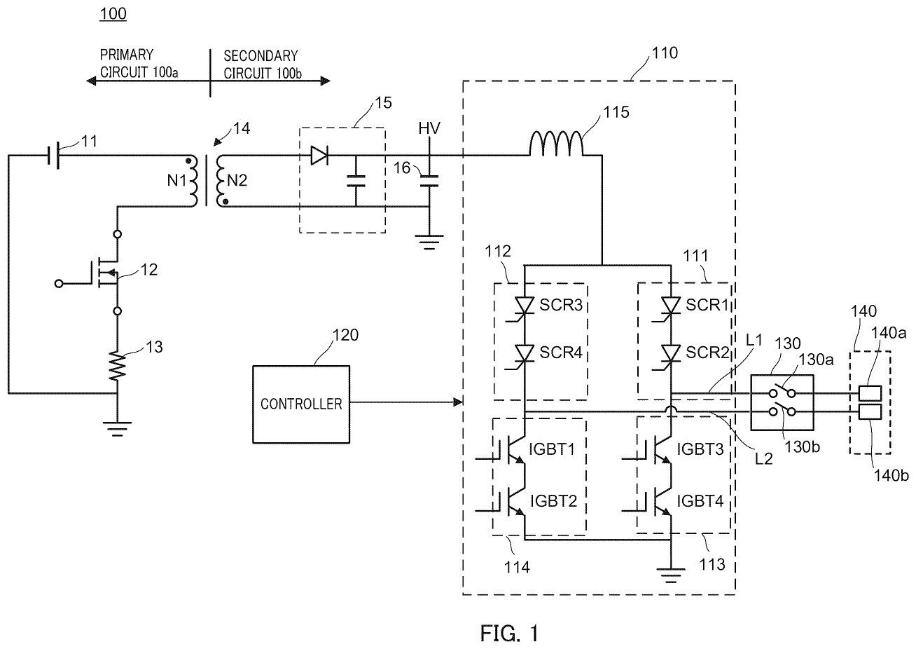

FIG. 1 is a circuit diagram illustrating the schematic configuration of defibrillator 100 according to an embodiment of the present disclosure. In particular, FIG. 1 is a circuit diagram illustrating a circuit portion of defibrillator 100 defibrillator 100 related to charging and discharging energy.

Defibrillator 100 includes primary circuit 100a and secondary circuit 100b. Primary circuit 100a includes battery 11, power MOSFET 12, resistor 13, and the like. Primary circuit 100a and secondary circuit 100b are connected to each other by transformer 14, and a high voltage (for example, about 2000 [V] to 2300 [V] in a case of a biphasic pulse) is output to the secondary circuit 100b side by transformer 14. Specifically, power is stored in transformer 14 including winding N1 and secondary winding N2 while power MOSFET 12 is on, and when power MOSFET 12 is switched to off, the stored power is output all at once to the secondary circuit 100b side by using a back electromotive force of transformer 14.

High-voltage capacitor 16 having a large capacitance is charged with the power output from transformer 14 via rectifier circuit 15. High-voltage capacitor 16 can store a charge of a high voltage (for example, 2000 [V]) compared to the voltage (for example, 24 [V]) of battery 11.

Biphasic pulse generation circuit 110 is connected to the rear stage side of high-voltage capacitor 16. Biphasic pulse generation circuit 110 can output a pulse of a first phase and then output a pulse of a second phase opposite to the first phase. As a result, it is possible to perform defibrillation with low energy and without inducing new ventricular fibrillation as compared with a defibrillator that outputs a monophasic pulse.

Biphasic pulse generation circuit 110 of the present embodiment is configured of an H-bridge type circuit. As described in PTL 1 and PTL 2, an H-bridge circuit is often used as a biphasic pulse generation circuit of a defibrillator.

Biphasic pulse generation circuit 110 includes first switch 111, second switch 112 that is connected in parallel to first switch 111, third switch 113 that is connected to the rear stage side of first switch 111 (third switch 113 is connected in series to first switch 111), and fourth switch 114 that is connected to the rear stage side of second switch 112 (fourth switch 114 is connected in series to second switch 112). The ON and OFF of first to fourth switches 111 to 114 is controlled by a control signal from controller 120.

In biphasic pulse generation circuit 110, inductor 115 is connected between high-voltage capacitor 16 and biphasic pulse generation circuit 110.

Biphasic pulse generation circuit 110 outputs a biphasic pulse from first output line L1 connected to a connection intermediate point between first switch 111 and third switch 113 and from second output line L2 connected to a connection intermediate point between second switch 112 and fourth switch 114.

Conduction control relay 130 is connected to first and second output lines L1 and L2 of biphasic pulse generation circuit 110. Electrode pad/paddle 140 is connected to the rear stage side of conduction control relay 130. More specifically, the connection intermediate point between first switch 111 and third switch 113 is connected to one electrode in electrode pad/paddle 140 via conduction control relay 130. In addition, the connection intermediate point between second switch 112 and fourth switch 114 is connected to the other electrode in electrode pad/paddle 140 via conduction control relay 130.

The ON/OFF of conduction control relay 130 and power MOSFET 12 is also controlled by controller 120.

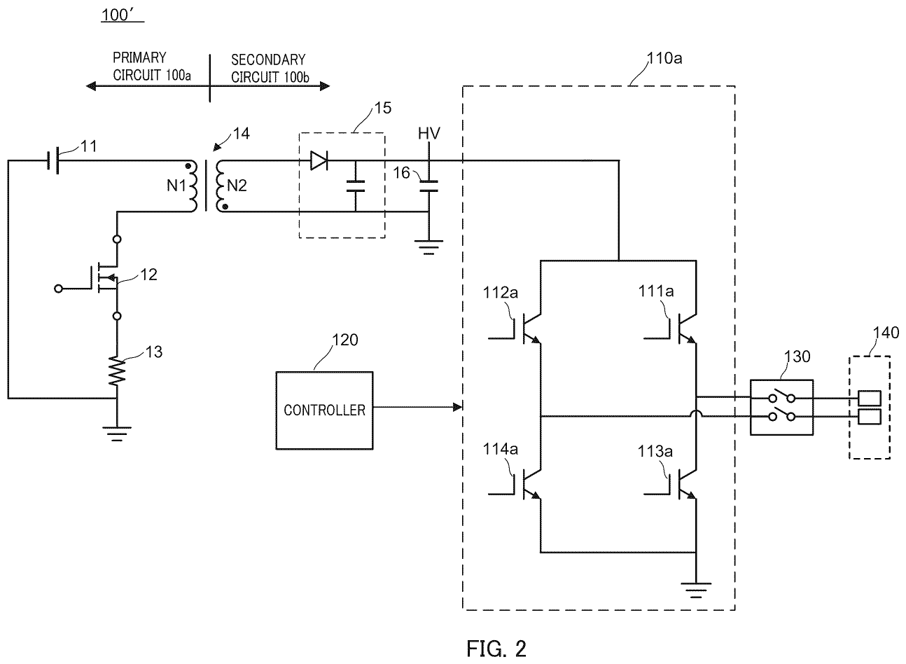

FIG. 2, in which portions corresponding to those in FIG. 1 are given the same reference numerals, illustrates a configuration example of defibrillator 100′ of the comparative example for defibrillator 100 of the present embodiment. A configuration the same as that of defibrillator 100′ is described in, for example, PTL 1 and PTL 2. In defibrillator 100′, an insulated gate bipolar transistor (IGBT) capable of withstanding the high voltage is used as switches 111a to 114a. In defibrillator 100′, each of four switches 111a to 114a of biphasic pulse generation circuit 110a is configured of one IGBT capable of withstanding a high voltage.

Compared to the configuration of FIG. 2, in biphasic pulse generation circuit 110 of the present embodiment illustrated in FIG. 1, first switch 111 is configured such that two thyristors SCR1 and SCR2 are connected in series, and second switch 112 is configured such that two thyristors SCR3 and SCR4 are connected in series. In addition, third switch 113 is configured such that two IGBT 3 and IGBT 4 are connected in series, and fourth switch 114 is configured such that two IGBT 1 and IGBT 2 are connected in series.

As described above, by configuring each of the first to fourth switches 111 to 114 in biphasic pulse generation circuit 110 in such a way that two or more thyristors or IGBTs are connected in series, the first to fourth switches 111 to 114 can be configured with inexpensive circuits having low withstand voltage performance as compared with a case where each of the first to fourth switches 111 to 114 is configured of one IGBT as illustrated in FIG. 2.

That is, in the circuit in the related art as illustrated in FIG. 2, a high voltage withstanding element that can withstand a voltage of about 2000 V is required as each IGBT, whereas in the configuration of FIG. 1, each thyristor and IGBT can use an element that can withstand about half that voltage. Therefore, in the configuration of FIG. 1, biphasic pulse generation circuit 110 can be configured using a low-voltage withstanding component as compared with the configuration of FIG. 2, and the product cost can be reduced as compared with the related art.

The comparison for the number of components constituting the first to fourth switches of the biphasic pulse generation circuit shows that biphasic pulse generation circuit 110 of the present embodiment illustrated in FIG. 1 requires a total of eight components of four thyristors and four IGBTs, whereas the number of components in biphasic pulse generation circuit 110a in the related art illustrated in FIG. 2 is four of the IGBTs. Therefore, biphasic pulse generation circuit 110 of the present embodiment requires almost twice the number of components in biphasic pulse generation circuit 110a in the related art. However, for example, components having a withstand voltage specification of about 1000 V are readily available in the market and can be obtained at a cost far lower than half the acquisition cost of components having a withstand voltage specification of 2000 V or more, thereby preventing the increase in the total product costs. Therefore, when the configuration of the present embodiment is adopted, the number of components increases as compared with the configuration in the related art, but the product costs decreases.

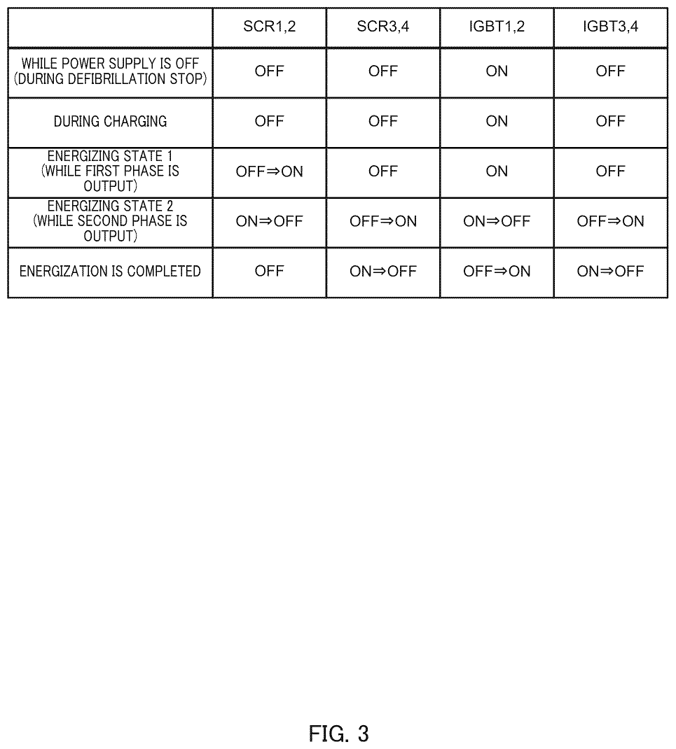

FIG. 3 is a diagram for describing an example of the ON/OFF operations of switches 111 to 114 of biphasic pulse generation circuit 110 of defibrillator 100.

During defibrillation stop (that is, while a power supply of defibrillator 100 is off) and during charging (that is, while high-voltage capacitor 16 is charged with defibrillation energy), IGBT 1 and IGBT 2, namely fourth switch 114, are set to an ON state, and SCR1 and SCR2, namely first switch 111, SCR3 and SCR4, namely second switch 112, and IGBT 3 and IGBT 4, namely third switch 113, are set to an OFF state. As a result, during defibrillation stop and during charging, no current flows through biphasic pulse generation circuit 110.

Defibrillator 100 transitions to an energized state when the charging is completed. The energized state includes a state in which the first phase is output (energizing state 1 in FIG. 3) and a state in which the second phase opposite to the first phase is output (energizing state 2 in FIG. 3). In the energizing state, conduction control relay 130 is controlled to be on.

In the energizing state 1, SCR 1 and SCR 2, namely first switch 111, are controlled to be switched from OFF to ON. As a result, the charge stored in high-voltage capacitor 16 flows in the following order: first switch 111 (SCR 1 and SCR 2) to conduction control relay 130a to first electrode 140a of electrode pad/paddle 140 to second electrode 140b of electrode pad/paddle 140 to conduction control relay 130b to fourth switch 114 (IGBT 1 and IGBT 2) to the ground.

In the energizing state 2, SCR 1 and SCR 2, namely first switch 111, are controlled to be switched from ON to OFF, SCR 3 and SCR 4, namely second switch 112, are controlled to be switched from OFF to ON, IGBT 1 and IGBT 2, namely fourth switch 114, are controlled to be switched from ON to OFF, and IGBT 3 and IGBT 4, namely third switch 113, are controlled to be switched from OFF to ON. As a result, the charge stored in high-voltage capacitor 16 flows in the following order: second switch 112 (SCR 3 and SCR 4) to conduction control relay 130b to second electrode 140b of electrode pad/paddle 140 to first electrode 140a of electrode pad/paddle 140 to conduction control relay 130a to the third switch (IGBT 3 and IGBT 4) to the ground.

As described above, between the energizing state 1 and the energizing state 2, the direction of the current flowing through electrode pad/paddle 140 is reversed, and the supply of the biphasic pulse to a patient is implemented.

For outputting the pulse of the first phase, fourth switch 114 is turned on and then first switch 111 is turned on, and for outputting the pulse of the second phase, third switch 113 is turned on and then second switch 112 is turned on.

In defibrillator 100 of the present embodiment illustrated in FIG. 1, by connecting a plurality of thyristors or a plurality of IGBTs in series in corresponding one of the first to fourth switches 111 to 114 constituting biphasic pulse generation circuit 110, a lower-voltage withstanding component than the thyristor or the IGBT in the related art can be used.

However, in the defibrillator, when each of the first to fourth switches 111 to 114 is configured by simply connecting a plurality of thyristors or a plurality of IGBTs in series, for example, due to variation in turn-on period caused by variation in components, the thyristor or the IGBT may be damaged or cannot be operated. Hereinafter, a configuration in consideration of the above point will be described.

### <2>Study and Configuration Related to Series Connection of Thyristors

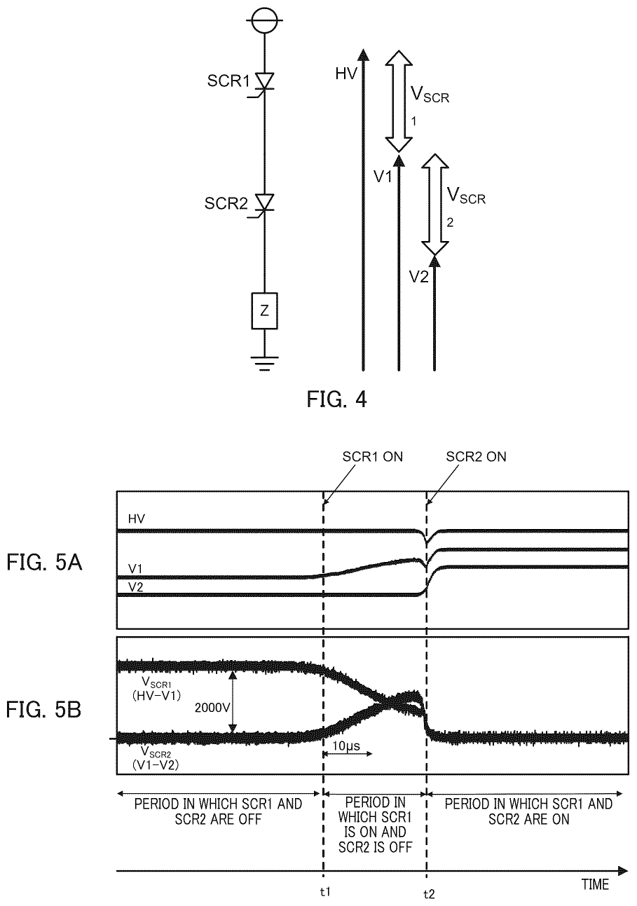

FIG. 4 illustrates a configuration in which two thyristors SCR1 and SCR2 are simply connected in series, as a reference example. Here, the configuration in which thyristors SCR1 and SCR2 are connected in series will be described, which can be applied to a configuration in which thyristors SCR3 and SCR4 are connected in series in the same manner.

Among the reference numerals illustrated on the right side of FIG. 4, HV indicates the potential of high-voltage capacitor 16 (see FIG. 1), V1 indicates the potential of thyristor SCR1, V2 indicates the potential of thyristor SCR2, VSCR1 indicates the voltage applied to thyristor SCR1, and VSCR2 indicates the voltage applied to thyristor SCR2. Z on the rear stage side of thyristor SCR2 indicates the biological impedance of a patient.

FIGS. 5A and 5B illustrate the potential and voltage values when thyristors SCR1 and SCR2 are turned on and off when the configuration of FIG. 4 is adopted. FIG. 5A illustrates the potential, and FIG. 5B illustrates the voltages applied to thyristors SCR1 and SCR2.

In FIGS. 5A and 5B, a period before time point t1 is a state in which both thyristors SCR1 and SCR2 are off (corresponding to a state of “defibrillation stop” and “charging” in FIG. 3), and a period after time point t2 is a state in which both thyristors SCR1 and SCR2 are on (corresponding to a state of “energizing state (while the first phase is output)” in FIG. 3).

In addition, a period between time point t1 and time point t2 is a state in which thyristor SCR1 is on and thyristor SCR2 is off. That is, time point t1 is the timing at which thyristor SCR1 is switched from OFF to ON, and time point t2 is the timing at which thyristor SCR2 is switched from OFF to ON.

Ideally, thyristor SCR1 and thyristor SCR2 are turned on at the same time, but in reality, the ON timing of thyristor SCR1 and the ON timing of thyristor SCR2 deviate slightly from each other. This deviation is illustrated in FIGS. 5A and 5B.

Here, since thyristors SCR1 and SCR2 are connected in series, the voltage VSCR1 applied to thyristor SCR1 is HV-V1, and the voltage VSCR2 applied to thyristor SCR2 is V1-V2.

As can be seen from FIG. 5B, in a state in which both thyristors SCR1 and SCR2 are off (steady state), 100% of the potential HV of high-voltage capacitor 16 is applied to thyristor SCR1, and when the voltage HV of high-voltage capacitor 16 is 2000 [V], the voltage VSCR1 applied to thyristor SCR1 becomes 2000 [V].

As described above, even when an attempt is made to lower the voltage applied to one thyristor by voltage division effect, that is by connecting thyristors SCR1 and SCR2 in series, the voltage division effect cannot be obtained when both thyristors SCR1 and SCR2 are in the OFF state (steady state), and thus the entire voltage is applied to one thyristor SCR1. As a result, it is not possible to use a thyristor having low withstand voltage performance.

### <2-1>Configuration Example 1

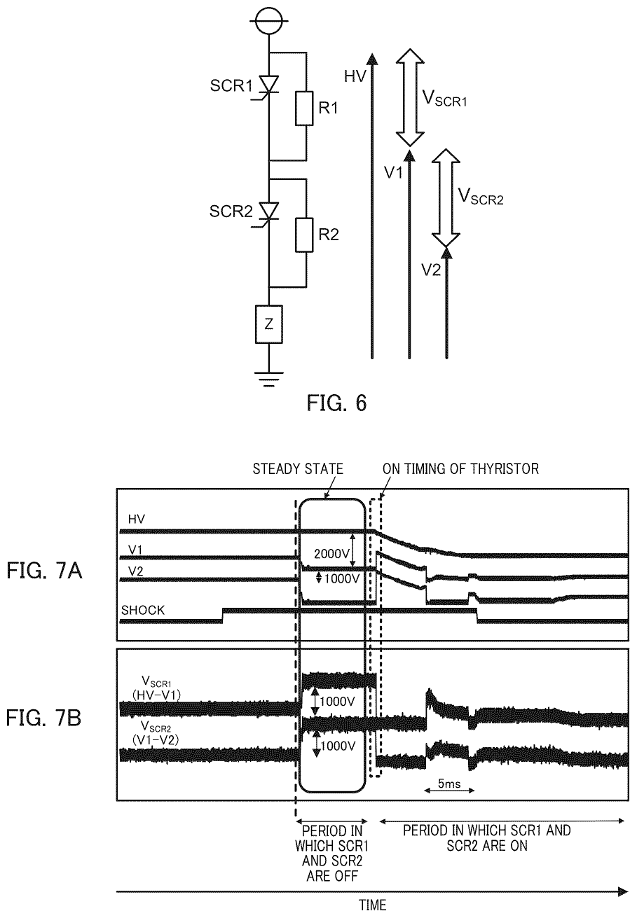

FIG. 6 illustrates a configuration example 1 of a portion related to the thyristors of the present embodiment based on the above study. Here, the configuration related to thyristors SCR1 and SCR2 will be described, which can be applied to a configuration related to thyristors SCR3 and SCR4 in the same manner.

In the configuration example 1, a resistor (voltage division resistor) R1 is connected in parallel to thyristor SCR1, and a resistor (voltage division resistor) R2 is connected in parallel to thyristor SCR2. As a result, even in a state in which both thyristors SCR1 and SCR2 are off (steady state), the voltage HV of high-voltage capacitor 16 is divided by resistors R1 and R2, so that it is possible to prevent a large voltage from being applied only to thyristor SCR1. As a result, it is possible to use a thyristor having low withstand voltage performance. Resistance values of resistors R1 and R2 are, for example, about 1 to 2 [Mω].

FIGS. 7A and 7B illustrate the potential and voltage values when thyristors SCR1 and SCR2 are turned on or off when the configuration of FIG. 6 is adopted. FIG. 7A illustrates the potential, and FIG. 7B illustrates the voltages applied to thyristors SCR1 and SCR2.

FIGS. 7A and 7B illustrate the potential and the voltage for a longer time than FIGS. 5A and 5B. A region illustrated as “steady state” surrounded by a frame line in FIGS. 7A and 7B is a state in which both thyristors SCR1 and SCR2 are off, and corresponds to a period before time point t1, which is indicated as problematic in FIGS. 5A and 5B. In addition, time points t1 and t2 of FIGS. 5A and 5B are present in a region illustrated as “ON timing of thyristor” surrounded by a dotted line.

As can be seen from FIG. 7B, in the configuration example 1, even in a state in which both thyristors SCR1 and SCR2 are off (steady state), the voltage HV (2000 [V]) of high-voltage capacitor 16 is divided by resistors R1 and R2, and the voltage VSCR1 applied to thyristor SCR1 and the voltage VSCR2 applied to thyristor SCR2 are each 1000 [V]. As a result, it is possible to use a thyristor having low withstand voltage performance.

By using resistors having a very large resistance value of 1 to 2 [MΩ] as resistors R1 and R2, a leakage current via resistors R1 and R2 can be made very small. Even when a leakage current occurs in the steady state, conduction control relay 130 is off in the steady state, so that the leakage current does not flow to a patient.

### <2-2>Configuration Example 2

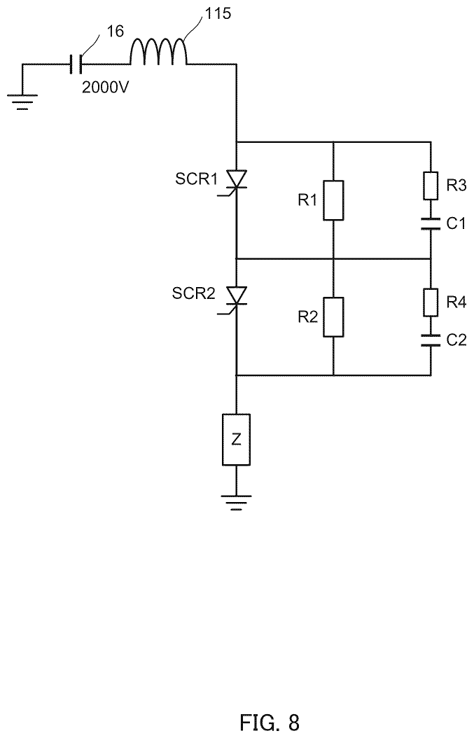

FIG. 8 illustrates a configuration example 2 of a portion related to the thyristors of the present embodiment. Here, the configuration related to thyristors SCR1 and SCR2 will be described, which can be applied to a configuration related to thyristors SCR3 and SCR4 in the same manner.

In the configuration example 2, in addition to the configuration example 1 illustrated in FIG. 6, an RC circuit in which resistor R3 and capacitor C1 are connected in series is connected in parallel to the resistor (voltage division resistor) R1 (the RC circuit may be said to be connected in parallel to thyristor SCR1). In addition, an RC circuit in which resistor R4 and capacitor C2 are connected in series is connected in parallel to the resistor (voltage division resistor) R2 (the RC circuit may be said to be connected in parallel to thyristor SCR2).

In addition, in the configuration example 2, inductor 115 is connected between high-voltage capacitor 16 and thyristors SCR1 and SCR3, that is, at an input stage of thyristors SCR1, SCR2, SCR3, and SCR4.

As a result, even when the timing at which thyristor SCR1 is turned on and the timing at which thyristor SCR2 is turned on deviate from each other, it is possible to prevent a large voltage from being applied to one of the thyristors. Hereinafter, the reason for this will be described.

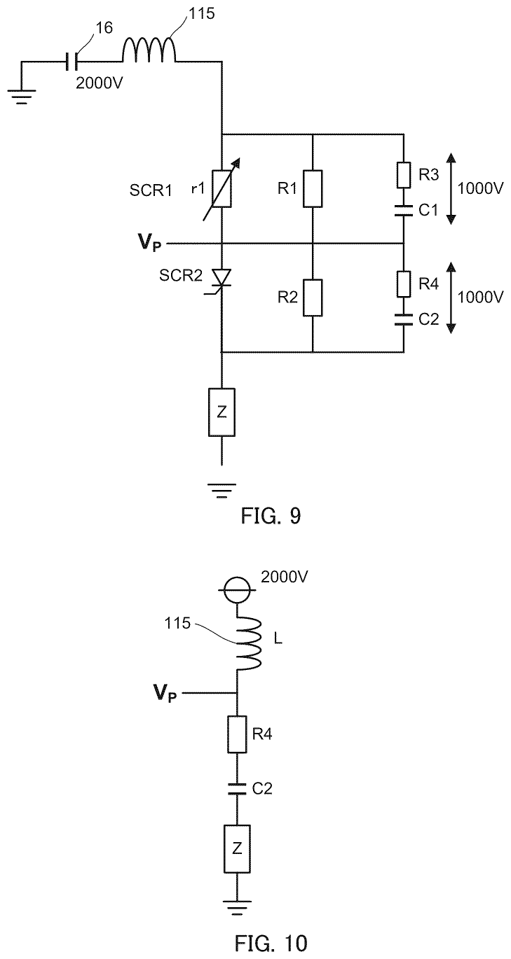

FIG. 9 is a circuit diagram when thyristor SCR1 is regarded as variable resistor r1 in the configuration example 2. Here, it is assumed that R1=R2=1 [M Ω], R3=R4=20 [Ω], C1=C2=1.5 [μF], and L=50 [μH].

As a worst condition, a case will be considered in which thyristor SCR1 is in a completely ON state and thyristor SCR2 is in a completely OFF state. In this case, r1 of FIG. 9 is substantially 0, and an OFF resistance value of thyristor SCR2 and the resistance value of R2 are sufficiently large with respect to the resistance value of R4 and can be ignored.

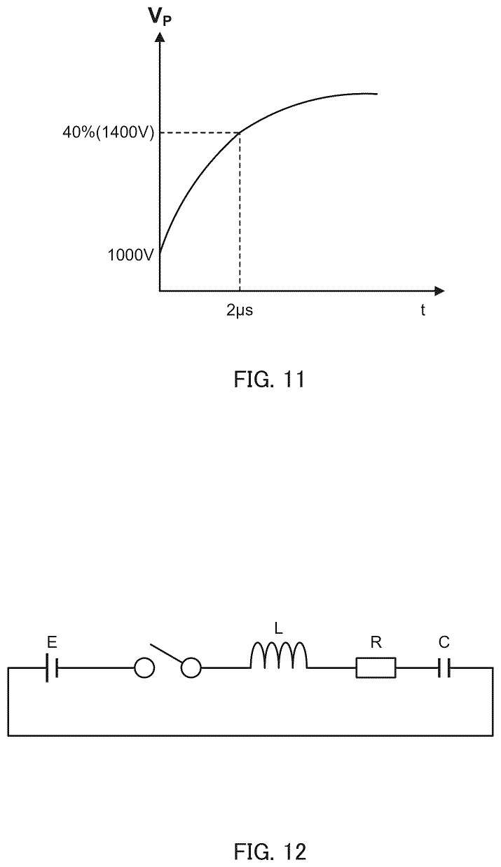

Therefore, the configuration of FIG. 9 can be rewritten as in FIG. 10. A voltage Vp on the rear stage side of inductor 115 (L) in the circuit configuration of FIG. 10 (that is, corresponding to the voltage applied to thyristor SCR1) changes over time t as illustrated in FIG. 11. The time t of FIG. 11 indicates an elapsed time from a state in which both thyristors SCR1 and SCR2 are off to a state in which only thyristor SCR1 is turned on. Here, in a steady state in which both thyristors SCR1 and SCR2 are off (that is, a state of t=0), the voltage Vp=1000 [V] as illustrated in the drawing due to the effect of resistors R1 and R2.

The voltage Vp gradually increases from immediately after thyristor SCR1 is turned on. This is an effect of providing inductor 115 (L), resistor R4, and capacitor C2, and when these components are not provided, the Vp instantly reaches 2000 [V] immediately after t=0.

As described above, by providing inductor 115 (L), resistor R4, and capacitor C2, even when the timing at which thyristor SCR1 is turned on and the timing at which thyristor SCR2 is turned on are slightly deviated from each other, damage to the thyristor can be prevented. For example, in the example of FIG. 11, when a thyristor having a withstand voltage of 1400 [V] is used, the damage to the thyristor can be prevented when the deviation in the timing of turning the thyristor on is within 2 [μs].

In the present embodiment, inductor 115 is connected between high-voltage capacitor 16 and biphasic pulse generation circuit 110, in addition to connecting the series circuit of the resistor and the capacitor in parallel to each of the plurality of thyristors SCR1 to SCR4. Therefore, the increase in the voltage applied to one of the thyristors is gentle when the ON timing deviates between the thyristors, preventing the damage to the thyristor.

The present disclosure is not limited to thereto, and inductor 115 may be omitted. In this case, when the capacitance of the capacitor is increased, the same effect as that of the embodiment can be obtained. However, increasing the capacitance of the capacitor leads to an increase in the circuit scale compared to the case where the inductor 115 is provided, and therefore the configuration of the embodiment is more preferable.

Here, a time response of voltage sharing of the elements in FIG. 10 was investigated. For the investigation, the circuit of FIG. 10 was simulated by the LCR series circuit of FIG. 12, and the behavior of the circuit after the switch was turned on was obtained by the following differential equation.

$$E = L \frac{di ( t )}{dt} + Ri ( t ) + \frac{1}{c} \int i ( t ) dt ( E = 2000 [ V ] , Vc ( 0 ) = 1000 [ V ] ) \qquad [ Equation 1 ]$$

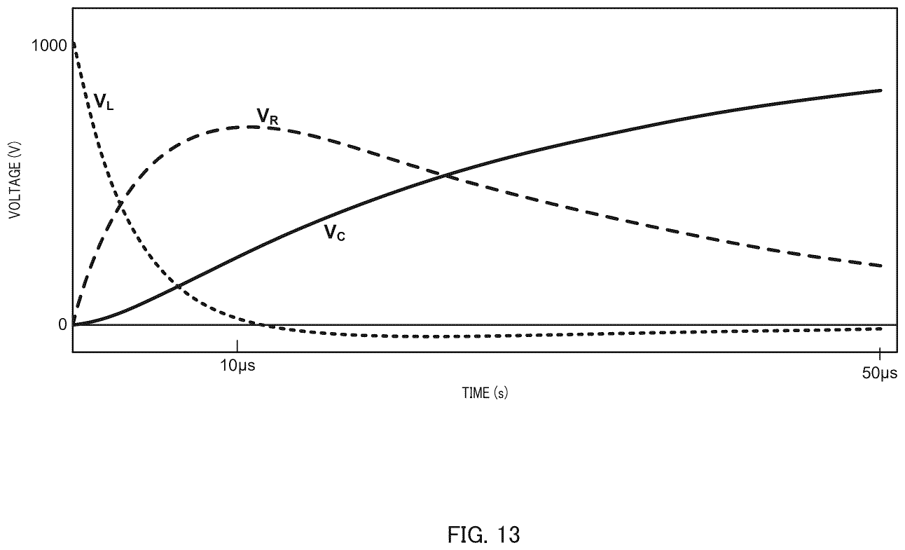

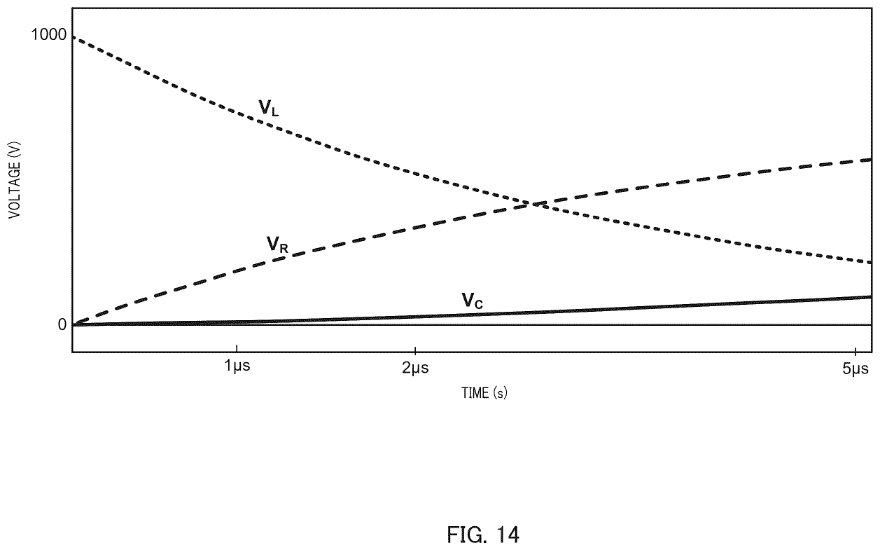

FIGS. 13 and 14 illustrate a time response of the voltage sharing of the elements based on a solution of the differential equation. VL indicates voltage sharing of the inductor L, VR indicates voltage sharing of resistor R, and VC indicates voltage sharing of capacitor C.

FIG. 13 illustrates a time response in a period of 0 to 50 [μs], that is, in a period of 50 [μs] from the moment the switch of FIG. 12 is turned on. FIG. 14 illustrates a time response in a period of 0 to 5 [μs], that is, in a period of 5 [μs] from the moment the switch of FIG. 12 is turned on.

As can be seen from FIG. 14, in a period of 0 to 2 [μs], the inductor L is predominant in the voltage sharing. Therefore, it is very effective to provide inductor 115 in terms of preventing the rapid increase in voltage immediately after the deviation in the ON timing of the thyristors.

### <3>Configuration in Case of Series Connection of IGBTs

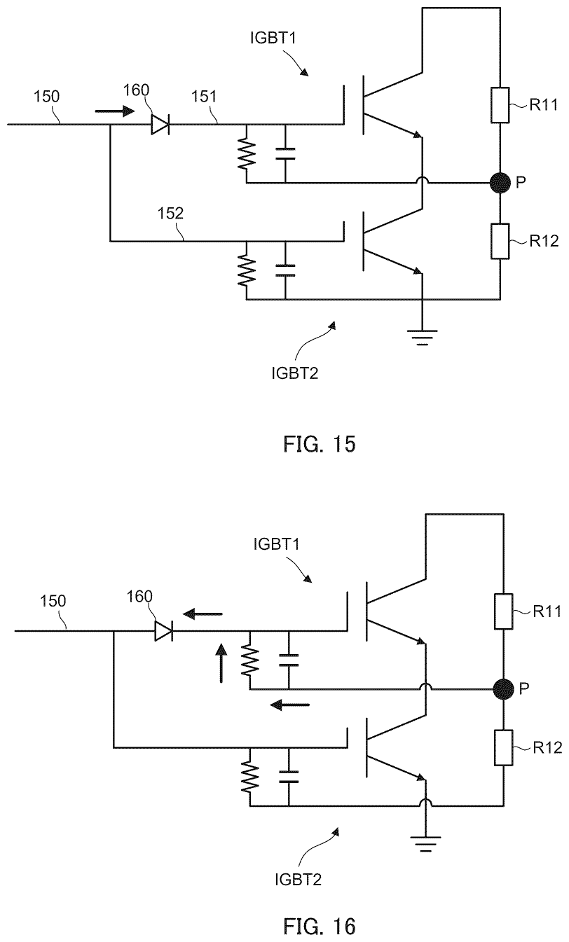

FIG. 15 illustrates a configuration example of a portion related to the IGBTs of the present embodiment. Here, the configuration related to IGBT 1 and IGBT 2 will be described, which can be applied to a configuration related to IGBT 3 and IGBT 4 in the same manner.

Branch signal lines 151 and 152 branched from common gate signal line 150 from controller 120 are respectively connected to gates of IGBT 1 and IGBT 2. Common gate signal line 150 is connected to controller 120 (FIG. 1). A control signal for controlling the ON and OFF of IGBT 1 and IGBT 2 is output from controller 120 via common gate signal line 150. The control signal is input to the gates of IGBT 1 and IGBT 2 via branch signal lines 151 and 152.

By providing common gate signal line 150 in this way, a control amount in controller 120 can be reduced as compared with a case where the control signals are output separately to IGBT 1 and IGBT 2 from controller 120. In addition, it is easy to synchronize the ON/OFF of the two IGBTs, namely IGBT 1 and IGBT 2, and the stability of the circuit is increased. The number of IGBTs connected to common gate signal line 150 is not limited to two, and may be two or more (that is, a plurality of IGBTs).

In addition, a signal delay amount of branch signal line 151 connected to IGBT 1 located on the front stage side among IGBT 1 and IGBT 2 is larger than a signal delay amount of branch signal line 152 connected to IGBT 2 located on the rear stage side among IGBT 1 and IGBT 2.

In the present embodiment, diode 160 as a delay circuit is provided in branch signal line 151, which makes the signal delay amount of branch signal line 151 larger than the signal delay amount of branch signal line 152. Alternatively, for example, the signal delay amount of branch signal line 151 may be made larger than the signal delay amount of branch signal line 152 by making the line length of branch signal line 151 longer than the line length of branch signal line 152.

In this way, by delaying the gate signal of IGBT 1 while providing common gate signal line 150 to IGBT 1 and IGBT 2, when the ON control signal is output from controller 120, IGBT 2 on the downstream side and IGBT 1 on the upstream side can be reliably turned on in the order of IGBT 2 and then IGBT 1, and reliability of the ON operation of IGBT 1 and IGBT 2 can be improved. As a result, when a plurality of IGBTs are connected to common gate signal line 150, the plurality of IGBTs can be controlled as if they were one IGBT.

Here, the control signal from controller 120 is a logic voltage, and is, for example, a voltage of about 5 V. On the other hand, the IGBT basically has the same characteristics as the FET and thus has a characteristic such that it cannot be turned on unless the gate signal is higher than the output line by a predetermined voltage. For example, when the voltage of the output line is 500 V, the IGBT cannot be turned on unless a control voltage of about 505 V is applied.

However, in the configuration in which IGBT 1 and IGBT 2 are connected in series, when the IGBT on the ground side is turned on first, the ON operation can be performed with a control voltage of about 5 V. In the present embodiment, this operation is implemented by providing diode 160 as the delay circuit.

In addition, even when IGBT 1 and IGBT 2 are connected in series, the same problem occurs as described in the study on the series connection of the thyristor as follows. Even when an attempt is made to lower the voltage applied to one of the IGBT by a voltage division effect, that is by connecting the two IGBT 1 and IGBT 2 in series, the voltage division effect cannot be obtained when both IGBT 1 and IGBT 2 are in the OFF state (steady state), and thus the entire voltage is applied to one IGBT. As a result, it is not possible to use an IGBT having low withstand voltage performance.

In consideration of the above point, as when the thyristors are connected in series, in IGBT 1 and IGBT 2 connected in series, resistor R11 is connected in parallel to IGBT 1, and resistor R12 is connected in parallel to IGBT 2, as illustrated in FIG. 15. As a result, it is possible to prevent a large voltage from being applied to only one IGBT by the voltage division effect of resistors R11 and R12. As a result, it is possible to use an IGBT having low withstand voltage performance. The resistance values of resistors R1 and R2 are, for example, about 1 to 2 [Mω].

In addition, by providing diode 160, it is possible to prevent a current from flowing into common gate signal line 150, as illustrated in FIG. 16. Specifically, in the configuration of FIG. 16, since the high voltage is divided by resistors R11 and R12, the voltage at a point P in the drawing is higher than a general logic voltage (control voltage). By providing diode 160, it is possible to prevent the adverse effect of the high voltage on the logic voltage (control voltage).

### <4>Other Embodiments

The embodiments described above are no more than specific examples in carrying out the present invention, and the technical scope of the present invention is not to be construed in a limitative sense due to the specific examples. That is, the present invention can be carried out in various forms without departing from the spirit and the main features thereof.

In the above-described embodiment, as illustrated in FIG. 1, first switch 111 is configured such that two thyristors SCR1 and SCR2 are connected in series, and second switch 112 is configured such that two thyristors SCR3 and SCR4 are connected in series. In addition, the embodiment describes a case in which third switch 113 is configured such that two IGBT 3 and IGBT 4 are connected in series and fourth switch 114 is configured such that two IGBT 1 and IGBT 2 are connected in series, but the present disclosure is not limited to thereto.

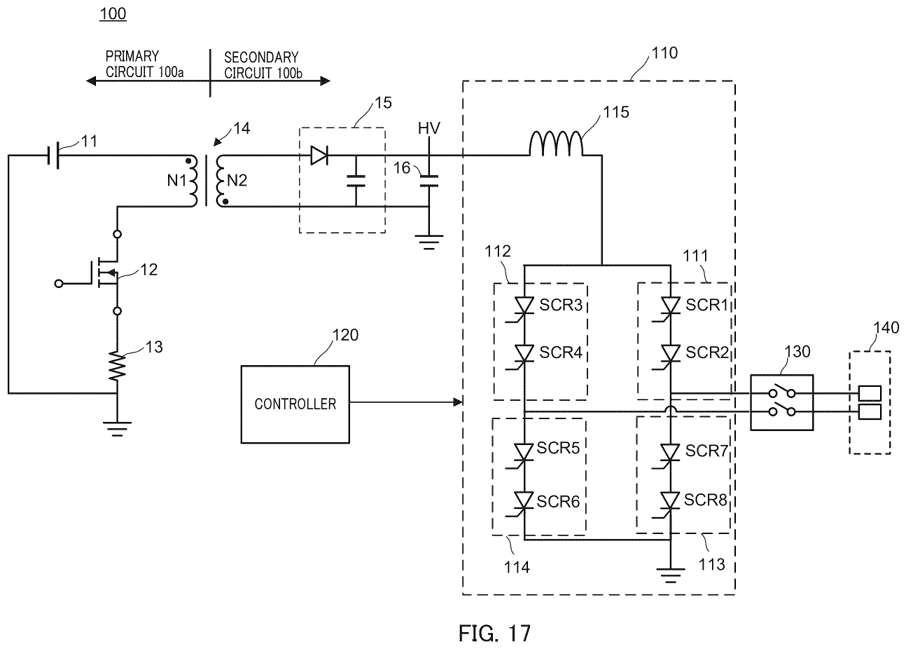

For example, as illustrated in FIG. 17, all of the first to fourth switches 111 to 114 may be configured such that the thyristors are connected in series. Even in this case, the configuration described in item <2>can be adopted in each of switches 111 to 114.

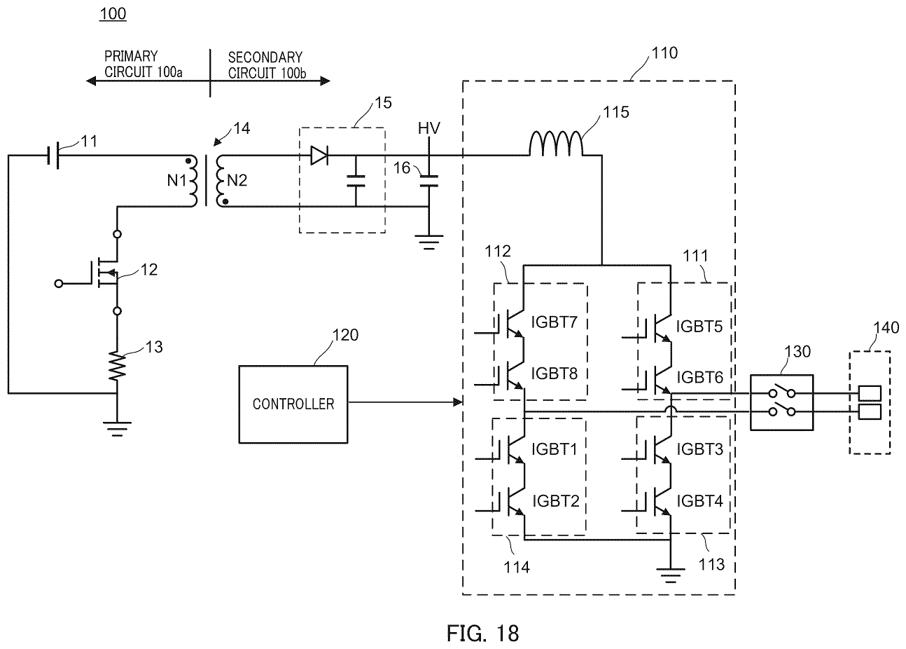

For example, as illustrated in FIG. 18, all of the first to fourth switches 111 to 114 may be configured such that the IGBTs are connected in series. Also in this case, the configuration described in item <3>can be adopted in each of switches 111 to 114.

In the above-described embodiment, a case where diode 160 is used as the delay circuit has been described, but a delay circuit other than the diode may be used.

### <5>Conclusion

(1) One aspect of defibrillator 100 of the present disclosure is a defibrillator

including a H-bridge type biphasic pulse generation circuit 110 connected to the rear stage side of high-voltage capacitor 16, in which: biphasic pulse generation circuit 110 includes first switch 111, second switch 112 that is connected in parallel to first switch 111, third switch 113 that is connected to the rear stage side of first switch 111 (third switch 113 is connected in series to first switch 111), and fourth switch 114 that is connected to the rear stage side of second switch 112 (fourth switch 114 is connected in series to second switch 112); biphasic pulse generation circuit 110 outputs a biphasic pulse from first output line L1 connected to a connection intermediate point between first switch 111 and third switch 113 and from second output line L2 connected to a connection intermediate point between second switch 112 and fourth switch 114; and in at least one of first switch 111 to fourth switch 114, a plurality of thyristors are connected in series, and resistors R1 and R2 are respectively connected in parallel to the plurality of thyristors.

As a result, even in a state in which both thyristors SCR1 and SCR2 connected in series are off (steady state), the voltage HV of high-voltage capacitor 16 is divided by resistors R1 and R2, so that it is possible to prevent a large voltage from being applied to only one of the thyristors, namely one thyristor SCR1. As a result, it is possible to use a thyristor having low withstand voltage performance. According to the above aspect, a defibrillator capable of using a lower-voltage withstanding component than that in the related art while favorably maintaining an original operation as a defibrillator can be realized.
(2) One aspect of defibrillator 100 of the present disclosure is as follows: in the defibrillator with configuration (1), a series circuit of the resistor and a capacitor is connected in parallel to each of the plurality of thyristors.

As a result, even when the timing at which one of the thyristors connected in series (thyristor SCR1) is turned on and the timing at which the other one of the thyristors (thyristor SCR2) is turned on deviate from each other, the rapid increase in voltage immediately after the deviation in the ON timing of the thyristors can be suppressed, and the application of a large voltage to the one thyristor SCR1 can be suppressed.
(3) One aspect of defibrillator 100 of the present disclosure is as follows: in the defibrillator with configuration (2), inductor 115 is connected between high-voltage capacitor 16 and biphasic pulse generation circuit 110.

As a result, the rapid increase in voltage immediately after the deviation in the ON timing of the thyristors can be effectively suppressed with a small circuit scale.
(4) One aspect of defibrillator 100 of the present disclosure is as follows: in the defibrillator with configuration (1), each of first switch 111 and second switch 112 has a configuration in which a plurality of thyristors are connected in series, and each of third switch 113 and fourth switch 114 has a configuration in which a plurality of insulated gate bipolar transistors (IGBTs) are connected in series.
(5) One aspect of defibrillator 100 of the present disclosure is as follows: in the defibrillator with configuration (4), branch signal lines 151 and 152 branched from common gate signal line 150 from controller 120 are respectively connected to gates of IGBT 1 and IGBT 2, and a signal delay amount of branch signal line 151 connected to IGBT 1 located on the front stage side among IGBT 1 and IGBT 2 is larger than a signal delay amount of branch signal line 152 connected to IGBT 2 located on the rear stage side among IGBT 1 and IGBT 2.

As a result, a processing amount of controller 120 can be reduced as compared with a case of separately controlling IGBT 1 and IGBT 2. In addition, it is easy to synchronize the ON/OFF of IGBT 1 and IGBT 2, and the stability of the circuit is increased. Furthermore, it is possible to reliably turn IGBT 2 (on the downstream side) on and then turn IGBT 1 (on the upstream side) on in this order, and it is possible to improve reliability of the ON operation when IGBT 1 and IGBT 2 are connected in series.
(6) One aspect of defibrillator 100 of the present disclosure is as follows: in the defibrillator with configuration (5), delay circuit 160 is provided in branch signal line 151 connected to IGBT 1 located on the front stage side.
(7) One aspect of defibrillator 100 of the present disclosure is as follows: in the defibrillator with configuration (5), resistors R11 and R12 are respectively connected in parallel to IGBT 1 and IGBT 2.

As a result, even in a state in which both IGBT 1 and IGBT 2 connected in series are off (steady state), the voltage HV of high-voltage capacitor 16 is divided by resistors R11 and R12, so that it is possible to prevent a large voltage from being applied to only one IGBT 1. As a result, it is possible to use an IGBT having low withstand voltage performance, as IGBT 1 and IGBT 2.
(8) One aspect of defibrillator 100 of the present disclosure is as follows: in the defibrillator with any one of configurations (1) to (7), biphasic pulse generation circuit 110 outputs a pulse of the first phase when the first and fourth switches 111 and 114 are in an ON state and the second and third switches 112 and 113 are in an OFF state; biphasic pulse generation circuit 110 outputs a pulse of the second phase opposite to the first phase when the first and fourth switches 111 and 114 are in an OFF state and the second and third switches 112 and 113 are in an ON state; for outputting the pulse of the first phase, fourth switch 114 is turned on and then first switch 111 is turned on; and for outputting the pulse of the second phase, third switch 113 is turned on and then second switch 112 is turned on.
(9) One aspect of defibrillator 100 of the present disclosure is a defibrillator including: a H-bridge type biphasic pulse generation circuit 110 connected to the rear stage side of high-voltage capacitor 16, in which: biphasic pulse generation circuit 110 includes first switch 111, second switch 112 that is connected in parallel to first switch 111, third switch 113 that is connected to the rear stage side of first switch 111 (third switch 113 is connected in series to first switch 111), and fourth switch 114 that is connected to the rear stage side of second switch 112 (fourth switch 114 is connected in series to second switch 112); biphasic pulse generation circuit 110 outputs a biphasic pulse from first output line L1 connected to a connection intermediate point between first switch 111 and third switch 113 and from second output line L2 connected to a connection intermediate point between second switch 112 and fourth switch 114; at least one of first switch 111 to fourth switch 114 has a configuration in which insulated gate bipolar transistors (IGBTs) 1 and IGBT 2 are connected in series; branch signal lines 151 and 152 branched from common gate signal line 150 from controller 120 are respectively connected to gates of IGBT 1 and IGBT 2; and a signal delay amount of branch signal line 151 connected to IGBT 1 located on a front stage side among IGBT 1 and IGBT 2 is larger than a signal delay amount of branch signal line 152 connected to IGBT 2 located on the rear stage side among IGBT 1 and IGBT 2.

As a result, a processing amount of controller 120 can be reduced as compared with a case of separately controlling IGBT 1 and IGBT 2. In addition, it is easy to synchronize the ON/OFF of IGBT 1 and IGBT 2, and the stability of the circuit is increased. Furthermore, it is possible to reliably turn IGBT 2 (on the downstream side) on and then turn IGBT 1 (on the upstream side) on in this order, and it is possible to improve reliability of the ON operation when IGBT 1 and IGBT 2 are connected in series.
(10) One aspect of defibrillator 100 of the present disclosure is as follows: in the defibrillator with configuration (9), a delay circuit (diode 160) is provided in branch signal line 151 connected to IGBT 1 located on the front stage side.
(11) One aspect of defibrillator 100 of the present disclosure is as follows: in the defibrillator with configuration (9), resistors R11 and R12 are respectively connected in parallel to IGBT 1 and IGBT 2.

As a result, even in a state in which both IGBT 1 and IGBT 2 connected in series are off (steady state), the voltage HV of high-voltage capacitor 16 is divided by resistors R11 and R12, so that it is possible to prevent a large voltage from being applied to only one IGBT 1. As a result, it is possible to use an IGBT having low withstand voltage performance as IGBT 1 and IGBT 2.

### REFERENCE SIGNS LIST

16 High-voltage capacitor
100 Defibrillator
110 Biphasic pulse generation circuit
111 to 114 Switch
115 Inductor
120 Controller
130 Conduction control relay
140 Electrode pad/paddle
150 Common gate signal line
151, 152 Branch signal line
160 Diode
SCR1 to SCR8 Thyristor
R1, R2, R3, R4, R11, R12 Resistor
C1, C2 Capacitor

What is claimed is:

1. A defibrillator comprising a biphasic pulse generation circuit connected to a rear stage side of a high-voltage capacitor, the biphasic pulse generation circuit being of an H-bridge type, wherein:
the biphasic pulse generation circuit includes
a first switch,
a second switch that is connected in parallel to the first switch,
a third switch that is connected to a rear stage side of the first switch, the third switch being connected in series to the first switch, and
a fourth switch that is connected to a rear stage side of the second switch, the fourth switch being connected in series to the second switch;
the biphasic pulse generation circuit outputs a biphasic pulse from a first output line connected to a connection intermediate point between the first switch and the third switch and from a second output line connected to a connection intermediate point between the second switch and the fourth switch; and
in at least one of the first switch to the fourth switch, a plurality of thyristors are connected in series and a resistor is connected in parallel to each of the plurality of thyristors.

2. The defibrillator according to claim 1, wherein
a series circuit of the resistor and a capacitor is connected in parallel to each of the plurality of thyristors.

3. The defibrillator according to claim 2, wherein
an inductor is connected between the high-voltage capacitor and the biphasic pulse generation circuit.

4. The defibrillator according to claim 1, wherein:
each of the first switch and the second switch has a configuration in which the plurality of thyristors are connected in series; and
each of the third switch and the fourth switch has a configuration in which a plurality of insulated gate bipolar transistors (IGBTs) are connected in series.

5. The defibrillator according to claim 4, wherein:
a plurality of branch signal lines branched from a common gate signal line from a controller are respectively connected to gates of the plurality of IGBTs; and
a signal delay amount of the branch signal line connected to an IGBT located on a front stage side among the plurality of IGBTs is larger than a signal delay amount of the branch signal line connected to an IGBT located on a rear stage side among the plurality of IGBTs.

6. The defibrillator according to claim 5, wherein
a delay circuit is provided in the branch signal line connected to the IGBT located on the front stage side.

7. The defibrillator according to claim 5, wherein
a resistor is connected in parallel to each of the plurality of IGBTs.

8. The defibrillator according to claim 1, wherein:
the biphasic pulse generation circuit outputs a pulse of a first phase when the first and fourth switches are in an ON state and the second and third switches are in an OFF state;
the biphasic pulse generation circuit outputs a pulse of a second phase opposite to the first phase when the first and fourth switches are in an OFF state and the second and third switches are in an ON state;
for outputting the pulse of the first phase, the first switch is turned on after the fourth switch is turned on; and
for outputting the pulse of the second phase, the second switch is turned on after the third switch is turned on.

9. A defibrillator comprising a biphasic pulse generation circuit connected to a rear stage side of a high-voltage capacitor, the biphasic pulse generation circuit being of an H-bridge type, wherein:
the biphasic pulse generation circuit includes
a first switch,
a second switch that is connected in parallel to the first switch,
a third switch that is connected to a rear stage side of the first switch, the third switch being connected in series to the first switch, and
a fourth switch that is connected to a rear stage side of the second switch, the fourth switch being connected in series to the second switch;
the biphasic pulse generation circuit outputs a biphasic pulse from a first output line connected to a connection intermediate point between the first switch and the third switch and from a second output line connected to a connection intermediate point between the second switch and the fourth switch;
at least one of the first switch to the fourth switch has a configuration in which a plurality of insulated gate bipolar transistors (IGBTs) are connected in series;
a plurality of branch signal lines branched from a common gate signal line from a controller are respectively connected to gates of the plurality of IGBTs; and
a signal delay amount of the branch signal line connected to an IGBT located on a front stage side among the plurality of IGBTs is larger than a signal delay amount of the branch signal line connected to an IGBT located on a rear stage side among the plurality of IGBTs.

10. The defibrillator according to claim 9, wherein
a delay circuit is provided in the branch signal line connected to the IGBT located on the front stage side.

11. The defibrillator according to claim 9, wherein
a resistor is connected in parallel to each of the plurality of IGBTs.
